(19) United States
(12) Patent Application Publication
(10) Pub. No.: US 2015/0282981 A1
(43) Pub. Date: Oct. 8, 2015

(54) Ophthalmic Drop Dispensing Tip, Dispenser and Method for Using Same

(71) Applicants: Richard C. FUISZ, Beverly Hills, CA (US); Joseph M. FUISZ, Surfside, FL (US)

(72) Inventors: Richard C. FUISZ, Beverly Hills, CA (US); Joseph M. FUISZ, Surfside, FL (US)

(21) Appl. No.: 14/247,673

(22) Filed: Apr. 8, 2014

(51) Int. Cl. A61F 9/00

(52) U.S. Cl. CPC A61F 9/0008

(57) ABSTRACT

A dispensing tip for dispensing drops of ophthalmic preparation includes a tip assembly having a tapered wall portion having tapered walls and an internal passage therethrough, the tapered walls narrowing from an area adjacent the first end of the tip assembly to a second end having a centrally located opening configured to dispense a drop of ophthalmic preparation that has passed through the passage. A recess is provided in the outer wall portion of the tip assembly, the recess extending around the tapered wall portion on planes substantially perpendicular to a longitudinal axis of the passage. A surface of the recess is configured to not be visible when the longitudinal axis of the passage is substantially aligned with an optical axis of an eye of the user. The surface of the recess has a marking to distinguish the surface from other portions of the tip assembly.

### BACKGROUND OF THE INVENTION

Self-administration of ophthalmic drops (eye drops) is often difficult for the user. Misalignment of the dropper or dispensing tip with the user's eye can cause the drop to partially miss the user's eye, resulting in less than the desired amount of the ophthalmic solution being administered, or often completely miss the user's eye, resulting in waste of the ophthalmic solution. In the alternative, a user may over-deliver solution anticipating misapplication in a “pray and spray” approach.

Various solutions to this problem have been proposed, ranging from an assembly that can be attached to the neck of an eye drop bottle or attached to the tip of an eye drop bottle and including various patterns that can be printed and used as optical targets to calibrate distance from the eye (U.S. Pat. No. 8,287,505 to Pine) to complicated alignment apparatus (see, e.g., U.S. Pat. No. 8,348,912 to Rehkemper et al.).

However, a more simple and effective ophthalmic drop dispensing tip, dispenser and method are still required.

### SUMMARY OF THE INVENTION

In one embodiment of the present invention, the invention relates to a dispensing tip for dispensing drops of ophthalmic preparation to an eye of a user. The dispensing tip includes a tip assembly having a first end configured to be connected to a container for holding ophthalmic preparation and having a tapered wall portion having tapered walls and an internal passage therethrough, the tapered walls narrowing from an area adjacent the first end of the tip assembly to a second end having a centrally located opening configured to dispense a drop of ophthalmic preparation that has passed through the passage. A recess is provided in the outer wall portion of the tip assembly, the recess extending around the tapered wall portion on planes substantially perpendicular to a longitudinal axis of the passage. A surface of the recess is configured to not be visible when the longitudinal axis of the passage is substantially aligned with an optical axis of an eye of the user. The surface of the recess has a marking to distinguish the surface from other portions of the tip assembly.

The present invention also relates to a dispensing bottle for dispensing drops of ophthalmic preparation to an eye of a user, the dispensing bottle including a container for holding ophthalmic preparation, and the dispensing tip of the present invention.

The present invention also relates to a method for dispensing drops of ophthalmic preparation to an eye of a user. The method includes providing the dispensing bottle of the present invention, the container having the ophthalmic preparation provided in the container, positioning the user's head such that the user's eyes are facing in an upward direction, positioning the dispensing bottle with the opening in the tip assembly facing an eye of the user, aligning the tip assembly until the surface of the recess is not visible; and then dispensing a drop of the ophthalmic preparation into the eye of a user.

In another embodiment of the present invention, the dispensing tip includes a tip assembly having a first end configured to be connected to a container for holding ophthalmic preparation and having a tapered wall portion having tapered walls and an internal passage therethrough, the tapered walls narrowing from an area adjacent the first end of the tip assembly to a second end having a centrally located opening configured to dispense a drop of ophthalmic preparation that has passed through the passage. A pair of spaced rings is provided in the outer wall portion of the tip assembly, each of the pair of rings extending around the tapered wall portion on planes substantially perpendicular to a longitudinal axis of the passage. The pair of spaced rings is configured such that both of the rings of the pair of rings are visible when the longitudinal axis of the passage is substantially aligned with an optical axis of an eye of the user. Each of the rings has a color different than the color of other portions of the tip assembly.

The present invention also relates to a dispensing bottle for dispensing drops of ophthalmic preparation to an eye of a user, the dispensing bottle including a container for holding ophthalmic preparation, and the dispensing tip of the present invention.

The present invention also relates to a method for dispensing drops of ophthalmic preparation to an eye of a user. The method includes providing the dispensing bottle of the present invention, the container having the ophthalmic preparation provided in the container, positioning the user's head such that the user's eyes are facing in an upward direction, positioning the dispensing bottle with the opening in the tip assembly facing an eye of the user, aligning the tip assembly until both of the rings of the pair of rings are visible; and then dispensing a drop of the ophthalmic preparation into the eye of a user.

### BRIEF DESCRIPTION OF THE DRAWINGS

FIG. 1 is side view of a first embodiment of the present invention.

FIG. 2A is a schematic end view of the first embodiment of the present invention when the longitudinal axis of the passage is not substantially aligned with an optical axis of an eye of the user.

FIG. 2B is a schematic end view of the first embodiment of the present invention when the longitudinal axis of the passage is substantially aligned with an optical axis of an eye of the user.

FIG. 3 is side view of a second embodiment of the present invention.

FIG. 4A is a schematic end view of the second embodiment of the present invention when the longitudinal axis of the passage is not substantially aligned with an optical axis of an eye of the user.

FIG. 4B is a schematic end view of the second embodiment of the present invention when the longitudinal axis of the passage is substantially aligned with an optical axis of an eye of the user.

### DETAILED DESCRIPTION OF THE PREFERRED EMBODIMENTS

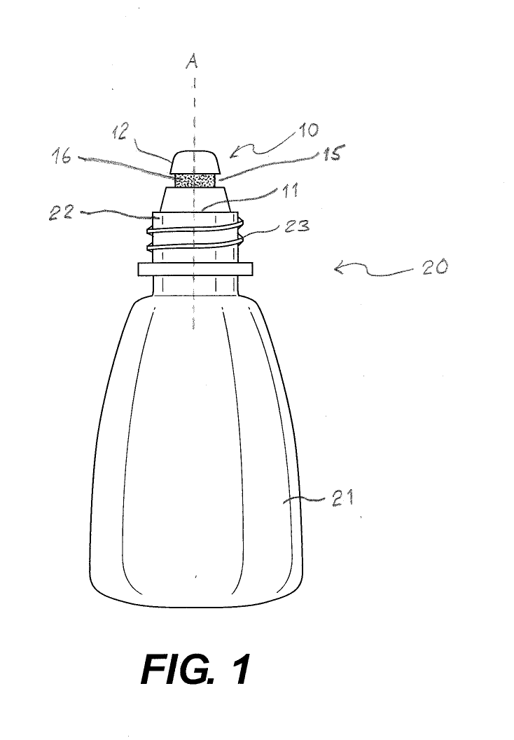

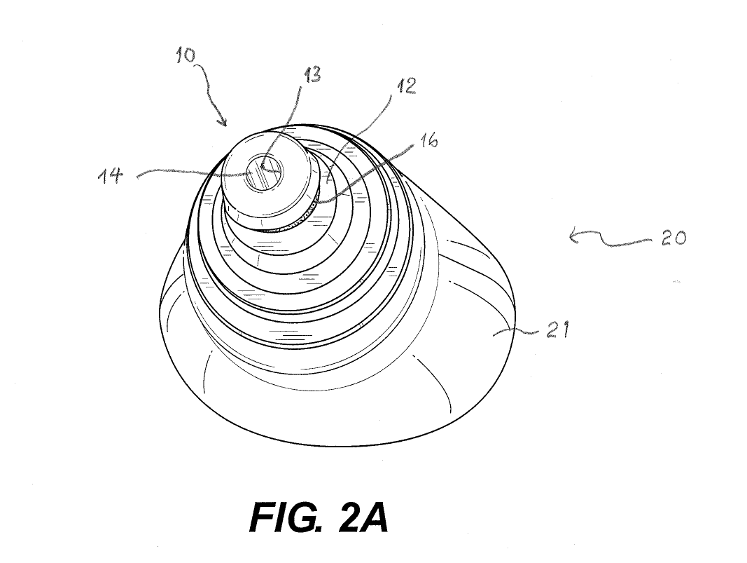

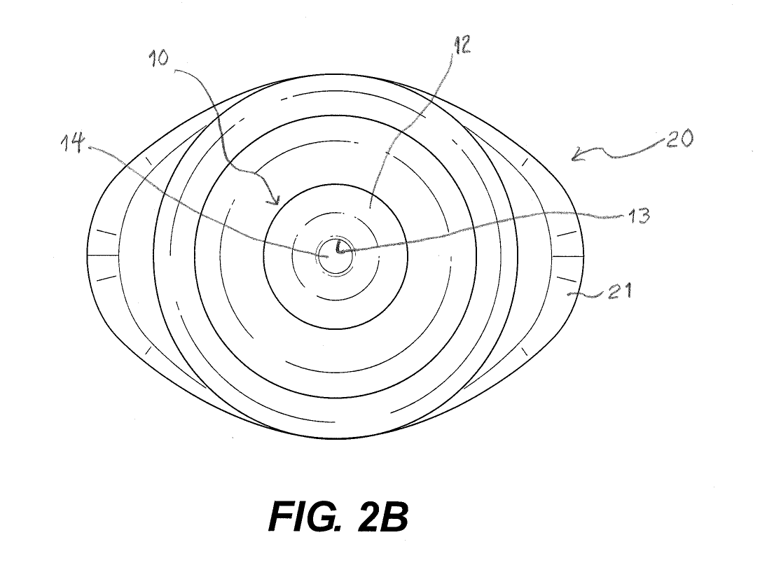

A first embodiment of the present invention is shown in connection with FIGS. 1, 2A and 2B. The invention relates both to a dispensing tip generally designated by the reference numeral 10 for dispensing drops of ophthalmic preparation to an eye of a user and to a dispensing bottle generally designated by the reference numeral 20 including a container 21 for holding ophthalmic preparation and the dispensing tip 10 of the present invention. By the phrase ophthalmic preparation, applicants are referring to any material or composition that can be dispensed into a user's eye. Ophthalmic preparations are generally, but not exclusively, sterile, liquid or semi-solid preparations that may contain one or more active pharmaceutical ingredient(s) intended for application to the eye. Different categories of ophthalmic preparations include, but are not limited to, drops consisting of emulsions, solutions or suspensions.

The dispensing tip 10 includes a tip assembly having a first end 11 configured to be connected to the container 21. The first end 11 can be connected to the container 21 by any means known in the art, e.g., by having a cylindrical extension (not shown) that fits within the neck 22 of the container. Alternatively, the first end 11 can be connected to the container 21 by being integral therewith. The dispensing tip 10 has a tapered wall portion 12 having tapered walls and an internal passage 13 therethrough, the tapered walls narrowing from an area adjacent the first end 11 of the tip assembly to a second end having a centrally located opening 14 configured to dispense a drop of ophthalmic preparation that has passed through the passage 13. A recess 15 is provided in the outer wall portion of the tip assembly, the recess extending around the tapered wall portion 12 on planes substantially perpendicular to a longitudinal axis A of the passage. A surface of the recess 15 is configured to not be visible when the longitudinal axis A of the passage 13 is substantially aligned with an optical axis of an eye of the user (the condition shown in FIG. 2B, as seen from the eye of the user). The surface of the recess 15 has a marking 16 to distinguish the surface from other portions of the tip assembly. The marking 16 is, e.g., a band having a color different than the color of other portions of the tip assembly.

The tapered wall portion may have a substantially frustoconical shape, as shown in the figures, but the shape is not limited thereto. The tapered wall portion may be hollow.

The neck 22 of the container, can include an externally threaded portion to cooperate with a cap (not shown) having cooperated internally threaded portion.

As is known in the art, the container bottle 21 may be made of a resilient material, such that when a user squeezes the container bottle 21, a drop of ophthalmic preparation is forced out of the opening 14 in the tip assembly 10.

FIG. 2A shows a condition of use (as seen from one eye of the user) in which the longitudinal axis A of the passage 13 is not substantially aligned with an optical axis of the eye of the user. In this condition, the eye of the user can see a portion of the colored band 16 in the recess 15. On the other hand, as shown in FIG. 2B, which shows a condition of use (as seen from one eye of the user), the colored band 16 in the surface of the recess 15 is not visible when the longitudinal axis A of the passage 13 is substantially aligned with an optical axis of an eye of the user.

In this embodiment, the marking 16 is shown as a contrasting color band and may be approximately 2 mm in thickness and may be placed near the end of the dispensing tip, preferably within 2 mm of the tip of the container. The color band is preferably a contrasting color compared to the color of the container and most preferably the container tip as well. The color band need be approximately 1-2 mm. down in the recess 15 lower than the wall 12 of the tip that proceeds to the opening. While these preferred characteristics are provided, the marking and the dimensions and location of the marking and recess can be changed as long as the eye of the user can see a portion of the marking 16 in the recess 15 when the longitudinal axis A of the passage 13 is not substantially aligned with an optical axis of the eye of the user, while the marking 16 in the surface of the recess 15 is not visible when the longitudinal axis A of the passage 13 is substantially aligned with an optical axis of an eye of the user.

While not being limited to any particular theory, the present invention is advantageous in use as follows. If one holds the eye drop container vertically over the eye, spreads the lids with the other hand and looks right at tip, most people will not be a sharp image at this distance; but this does not matter because, as the pupil of the eye stares at the tip, if the marking, e.g., colored band disappears, the pupil of the eye and the opening are lined up for easy application. Any notice of the band color can mean the container is not vertical or that the tip is not lined with the pupil.

The latter occurs because optically a lined up bottle held vertically will result in no sense of the color band at all and will result in a direct hit of the drop near the pupil. As the tip is off center to the pupil, part of the color band becomes visible.

It is also contemplated that the present invention may be sighted with respect to the pupil, but the tip applicator may be offset intentionally so as to direct the drop to the lower lid or other desired target site. The pupil is site reference point for the present invention in any case.

By substantially aligned with an optical axis of an eye of the user, applicants intend to include not only the condition of perfect alignment, but also alignments which are either or both some number of degrees tilted or some distance off-axis as long as a drop dispensed in a tilted or off-axis condition nevertheless is dispensed onto the pupil.

Thus, the present invention also relates to a method for dispensing drops of ophthalmic preparation to an eye of a user. The method includes providing the dispensing bottle 20 of the present invention, the container 21 having the ophthalmic preparation provided in the container 21, positioning the user's head such that the user's eyes are facing in an upward direction, positioning the dispensing bottle 21 with the opening 14 in the tip assembly facing an eye of the user, aligning the tip assembly 10 until the marked surface 16 of the recess 15 is not visible (the condition of FIG. 2B); and then dispensing a drop of the ophthalmic preparation into the eye of a user.

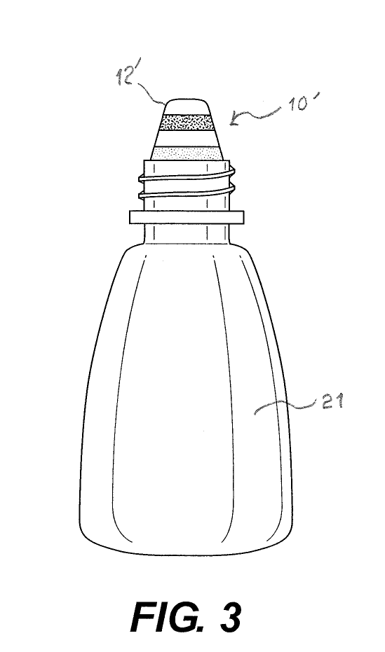

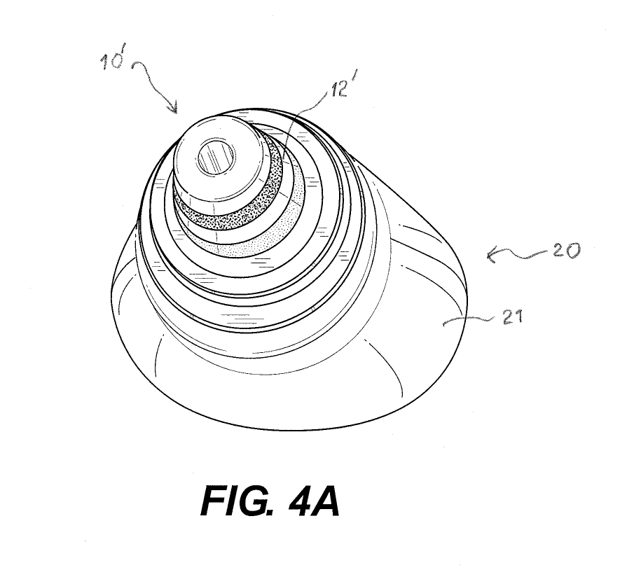

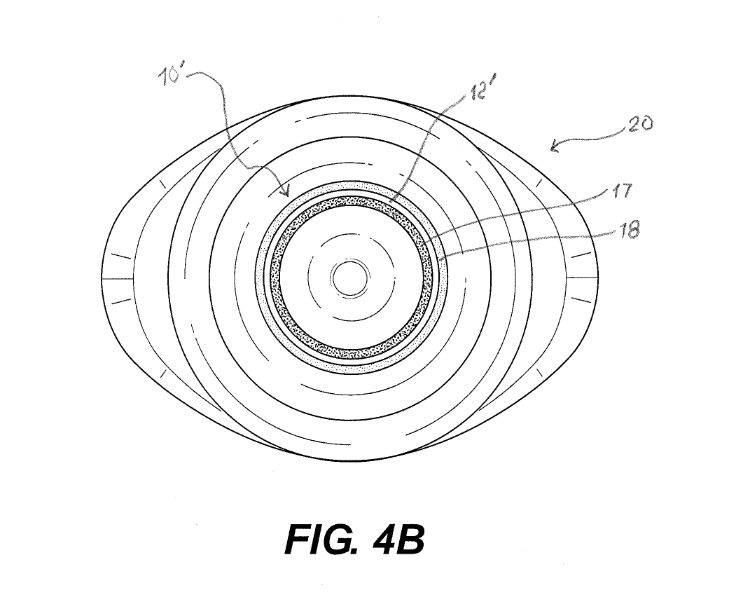

A second embodiment of the present invention is shown in connection with FIGS. 3, 4A and 4B. In this embodiment, the dispensing tip 10′ includes a tapered wall portion 12′ having tapered walls and an internal passage therethrough. A pair of spaced rings 17, 18 is provided in the outer wall portion 12 of the tip assembly 10, each of the pair of rings 17,18 extending around the tapered wall portion on planes substantially perpendicular to a longitudinal axis of the passage (see, A in FIG. 1). The pair of spaced rings 17, 18 is configured such that both of the rings 17, 18 of the pair of rings are visible when the longitudinal axis A of the passage is substantially aligned with an optical axis of an eye of the user, as shown in FIG. 4B. Each of the rings has a marking or color different than the marking or color of other portions of the tip assembly and from each other. In the condition shown in FIG. 4A, which shows a condition of use (as seen from one eye of the user) in which the longitudinal axis A of the passage 13 is not substantially aligned with an optical axis of the eye of the user, the eye of the user can see only portions of the rings 17, 18, i.e., the colored bands, do not appear as complete concentric rings. On the other hand, as shown in FIG. 4B, which shows a condition of use (as seen from one eye of the user), the rings 17, 18, i.e., the colored bands, appear as complete concentric rings; in this condition the longitudinal axis A of the passage 13 is substantially aligned with an optical axis of an eye of the user.

In this embodiment, the rings 17, 18 are not sunken but on the surface 12. One is preferably placed near the tip (1-2 mm distal) and a second non-sunken band placed preferably about 4-12 mm proximal (away from the tip). These spaced rings act like a gunsight so that if the bottle is vertical, a line up with the pupil would show 2 rings. While these preferred characteristics are provided, the dimensions, colors and location of the rings 17, 18 can be changed as long as the eye of the user can see only portions of the rings 17, 18 (i.e., the colored bands do not appear as complete concentric rings when the longitudinal axis A of the passage 13 is not substantially aligned with an optical axis of an eye of the user (FIG. 4A), while the rings 17, 18 appear as complete concentric rings when the longitudinal axis A of the passage 13 is substantially aligned with an optical axis of an eye of the user.

1. A dispensing tip for dispensing drops of ophthalmic preparation to an eye of a user, comprising:
a tip assembly having a first end configured to be connected to a container for holding ophthalmic preparation and having a tapered wall portion having tapered walls and an internal passage therethrough, the tapered walls narrowing from an area adjacent the first end of the tip assembly to a second end having a centrally located opening configured to dispense a drop of ophthalmic preparation that has passed through the passage; and
a recess provided in the outer wall portion of the tip assembly, the recess extending within the tapered walls on planes perpendicular to a longitudinal axis of the passage,
wherein a surface of the recess is configured to not be visible when the longitudinal axis of the passage is substantially aligned with an optical axis of an eye of the user and to have at least a portion visible when the longitudinal axis of the passage is not substantially aligned with an optical axis of an eye of the user, and wherein the surface of the recess has a marking to distinguish the surface from other portions of the tip assembly.

2. The dispensing tip according to claim 1, wherein the marking is a band having a color different than the color of other portions of the tip assembly.

3. The dispensing tip according to claim 1, wherein the tapered wall portion has a frustoconical shape.

4. The dispensing tip according to claim 1, wherein tapered wall portion is hollow.

5. A dispensing bottle for dispensing drops of ophthalmic preparation to an eye of a user, comprising:
a container for holding ophthalmic preparation;
a tip assembly having a first end operably connected to the container and having a tapered wall portion having tapered walls and an internal passage therethrough, the tapered walls narrowing from an area adjacent the first end of the tip assembly to a second end having a centrally located opening configured to dispense a drop of ophthalmic preparation that has passed through the passage; and
a recess provided in the outer wall portion of the tip assembly, the recess extending within the tapered walls on planes perpendicular to a longitudinal axis of the passage,
wherein a surface of the recess is configured to not be visible when the longitudinal axis of the passage is substantially aligned with an optical axis of an eye of the user and to have at least a portion visible when the longitudinal axis of the passage is not substantially aligned with an optical axis of an eye of the user, and wherein the surface of the recess has a marking to distinguish the surface from other portions of the tip assembly.

6. The dispensing bottle according to claim 5, wherein the marking is a band having a color different than the color of other portions of the tip assembly.

7. The dispensing bottle according to claim 5, wherein the tapered wall portion has a frustoconical shape.

8. The dispensing bottle according to claim 5, wherein tapered wall portion is hollow.

9. The dispensing bottle according to claim 5, wherein the container bottle is made of a resilient material such that when a user squeezes the container bottle, a drop of ophthalmic preparation is forced out of the opening in the tip assembly.

10-20. (canceled)

21. The dispensing bottle according to claim 5, wherein the tapered wall portion immediately adjacent the second end has a frustoconical shape.

22. The dispensing tip according to claim 1, wherein the tapered wall portion immediately adjacent the second end has a frustoconical shape.
